SEQUENCE LISTING (4 sequences)

SEQ ID NO: 1 - protein, 517 aa, unidentified
MDIIGGQHLRQMWDDLADVYGHKTALICESSGGVVNRYSYLELNQEINRTANLFYTLGIRKGDKVALHLDNCPEFIFCWFGLAKIGAIMVPINARLLCEESAWILQNSQACLLVTSAQFYPMYQQIQQEDATQLRHICLTDVALPADDGVSSFTQLKNQQPATLCYAPPLSTDDTAEILFTSGTTSRPKGVVITHYNLRFAGYYSAWQCALRDDDVYLTVMPAFHIDCQCTAAMAAFSAGATFVLVEKYSARAFWGQVQKYRATVTECIPMMIRTLMVQPPSANDQQHRLREVMFYLNLSEQEKDAFCERFGVRLLTSYGMTETIVGIIGDRPGDKRRWPSIGRVGFCYEAEIRDDHNRPLPAGEIGEICIKGIPGKTIFKEYFLNPQATAKVLEADGWLHTGDTGYRDEEDFFYFVDRRCNMIKRGGENVSCVELENIIAAHPKIQDIVVVGIKDSIRDEAIKAFVVLNEGETLSEEEFFRFCEQNMAKFKVPSYLEIRKDLPRNCSGKIIRKNLK

SEQ ID NO: 2 - DNA, 1554 bp, unidentified
  source 1..1554: mol_type = other DNA
atggatatcattggcggacaacatctacgtcaaatgtgggacgatcttgcggacgtttacggtcataaaacggcgctgatttgtgaatccagcggcggagtcgttaaccggtatagttatcttgagttaaatcaggagattaaccgcacggcaaacctgttttatacgctggggattcgcaaaggcgacaaggttgcactacatctcgacaactgcccggaatttatcttttgctggttcgggctggcaaaaattggcgcgattatggtgccgattaacgcccgcctgttgtgcgaggaaagcgcgtggatcctgcaaaatagccaggcgtgcctgctggtgaccagtgcgcaattctatcctatgtatcaacagattcagcaggaagatgccactcaattgcggcacatttgcctgacagatgtggcacttcccgctgatgatggcgtgagttcgtttactcaactgaaaaatcaacaacctgccaccttgtgctatgcaccgccgctatcgactgacgatacggcggaaattctcttcacctccggcaccacctcccgaccgaaaggtgtggtgattacccattacaacctgcgcttcgctggatattactccgcctggcagtgtgcactgcgtgacgatgacgtctacctgacggtaatgcctgcgtttcatatcgattgccagtgtactgcggcgatggcggcgttttctgccggggccacctttgtgctggtcgagaaatacagcgcccgcgccttctggggacaggtacagaagtaccgcgccaccgttaccgaatgtattccgatgatgatccgtacgttgatggtacagccgccttcagcgaacgatcagcaacaccgcctgcgggaagtgatgttttatctcaacttgtcggagcaggaaaaagatgcgttttgtgaacgcttcggcgttcgcttgctgacgtcttatgggatgacggaaaccattgtgggcattatcggcgatcgtcctggcgataaacgacgctggccgtcgattggtcgggtggggttttgctacgaagcggagatccgcgacgatcacaatcgcccgctcccggctggtgagatcggtgaaatctgcattaaaggcatacctgggaaaaccatcttcaaagagtactttctcaacccacaagccactgcgaaagtgctggaagccgatggctggctgcataccggcgataccggataccgcgacgaagaggactttttttatttcgtcgatcgccgctgcaatatgattaaacgtggcggcgagaatgtctcctgcgtggagctggaaaatattatcgccgcgcacccgaaaattcaggacatcgtggttgtgggtattaaagattcgattcgcgatgaagccatcaaagcatttgtggtgctgaatgaaggtgaaacattgagcgaagaggaatttttccgcttctgcgaacaaaatatggcgaaatttaaagtgccctcttatctggagatcagaaaagatctgccacgtaattgctcggggaaaataattagaaagaatctgaaataa

SEQ ID NO: 3 - DNA, 54 bp, unidentified
  source 1..54: mol_type = other DNA
agcaaatgggtcgcggatccgaattcatggatatcattggcggacaacatctac

SEQ ID NO: 4 - DNA, 61 bp, unidentified
  source 1..61: mol_type = other DNA
tggtgctcgagtgcggccgcaagctttttcagattctttctaattattttccccgagcaat

(19) United States
(12) Patent Application Publication
(10) Pub. No.: US 2024/0132925 A1
(43) Pub. Date: Apr. 25, 2024

(54) METHOD FOR PREPARING PYRROLIDONE

(71) Applicant: JIANGNAN UNIVERSITY, Wuxi (CN)

(72) Inventors: Jing WU, Wuxi (CN); Xuling JIANG, Wuxi (CN); Liming LIU, Wuxi (CN); Wei SONG, Wuxi (CN); Yiwen ZHOU, Wuxi (CN); Xiulai CHEN, Wuxi (CN); LIU Jia, Wuxi (CN); Cong GAO, Wuxi (CN)

(21) Appl. No.: 17/998,217

(22) PCT Filed: Oct. 18, 2022
(86) PCT No.: PCT/CN2022/126215

§ 371 Date: Nov. 8, 2022

(51) Int. Cl. C12P 17/10; C12N 9/00; C12N 15/70

(52) U.S. Cl. CPC C12P 17/10; C12N 9/93; C12N 15/70; C12R 2001/19; C12Y 602/01

(57) ABSTRACT

The invention provides a method for preparing pyrrolidone, and the invention provides a method for catalytically preparing pyrrolidone with γ-aminobutyric acid in the presence of carnitine-CoA ligase CaiC. The carnitine-CoA ligase CaiC has an amino acid sequence as shown in SEQ ID NO:1. The ligase has catalytic activity in the cyclization of γ-aminobutyric acid to produce pyrrolidone. The carnitine-CoA ligase provided in the present invention affords a yield of pyrrolidone of 3.26 g/L and a molar yield of 39.53% in 24 h when γ-aminobutyric acid is used as a substrate, thus reducing the production period, improving the production of pyrrolidone, and accelerating the industrialization process of producing pyrrolidone by enzymatic conversion method.

### FIELD OF THE INVENTION

The present invention relates to the technical field of bioengineering, and specifically to a method for preparing pyrrolidone.

### DESCRIPTION OF THE RELATED ART

Pyrrolidone, also known as butyrolactam or α-pyrrolidone, is a colorless crystal useful as a solvent and an intermediate in organic synthesis and as a precursor in the manufacture of various compounds such as nylon 4 and vinylpyrrolidone, thus having many important applications in industry. Pyrrolidone and its derivatives are five-membered nitrogen-containing heterocyclic molecules, which have some unique performances in terms of the biological activity. The molecular backbone of such heterocyclic compounds is found in many natural products.

At present, pyrrolidone is mainly synthesized by the chemical method, in which γ-butyrolactone and ammonia are reacted at a high temperature under a high pressure, to obtain the target product with a yield of 94%. However, the chemical method has a high yield, but large energy consumption and toxic adverse effects on the environment, thus not meeting the requirements of green production, safe production and sustainable development. Compared with the traditional chemical method, the preparation of pyrrolidone by biological method has the advantages of stable and safe product quality, mild process conditions, and environmental protection, to reduce the pressure on the environment and resources. Therefore, there is an urgent need for an effective biological method to efficiently produce pyrrolidone.

In recent years, some studies have been carried out on the biosynthesis of pyrrolidone in China and other countries. At present, the biosynthesis of pyrrolidone mainly includes microbial fermentation and enzymatic transformation. However, the microbial fermentation has a longer fermentation period and a low production intensity, thus being not suitable for industrial production. However, the existing enzymatic conversion has a low catalytic efficiency, and a very low yield. Therefore, there is an urgent need for an effective enzymatic conversion method to efficiently produce pyrrolidone.

### SUMMARY OF THE INVENTION

To solve the above technical problems, the present invention provides a method for preparing pyrrolidone. Specifically, the present invention provides a method for catalytically preparing pyrrolidone with γ-aminobutyric acid in the presence of carnitine-CoA ligase CaiC, or a method for producing pyrrolidone through whole-cell conversion of γ-aminobutyric acid by constructing a recombinant strain with the carnitine-CoA ligase CaiC. The present invention has advantages such as low damage to environment, short production period, and reduced by-products in the conversion, thus greatly improving the industrialized production efficiency.

A first object of the present invention is to provide a method for preparing pyrrolidone. The method comprises catalytically preparing pyrrolidone from γ-aminobutyric acid in the presence of carnitine-CoA ligase CaiC or a whole cell expressing carnitine-CoA ligase CaiC.

Preferably, the carnitine-CoA ligase CaiC has an amino acid sequence as shown in SEQ ID NO:1.

Preferably, the nucleotide sequence encoding the carnitine-CoA ligase CaiC is as shown in SEQ ID NO:2.

Preferably, the whole cell is obtained by collecting the recombinant strain expressing carnitine-CoA ligase CaiC after 12-16 h of induction by IPTG.

Preferably, the recombinant strain is produced with Escherichia coli as a host, and the carnitine-CoA ligase CaiC is expressed using PET-28a as an expression vector.

Preferably, the E. coli is Escherichia coli BL21 (DE3).

Preferably, the catalytic reaction system comprises γ-aminobutyric acid, ATP and Mg²⁺.

Preferably, in the reaction system, the whole cell has a final concentration of 15-25 g/L.

Preferably, in the reaction system, γ-aminobutyric acid has a final concentration of 5-15 g/L.

Preferably, the reaction system comprises 40-60 mM ATP and 20-40 mM Mg²⁺.

Preferably, the reaction system has a pH of 7.4-7.6, and the reaction temperature is 35-38° C.

### Beneficial Effects of the Present Invention

the present invention provides a method for catalytically preparing pyrrolidone with γ-aminobutyric acid in the presence of carnitine-CoA ligase CaiC. The carnitine-CoA ligase CaiC has an amino acid sequence as shown in SEQ ID NO:1. The ligase has catalytic activity in the cyclization of γ-aminobutyric acid to produce pyrrolidone. The carnitine-CoA ligase provided in the present invention affords a yield of pyrrolidone of 3.26 g/L and a molar yield of 39.53% in 24 h when γ-aminobutyric acid is used as a substrate, thus reducing the production period, improving the production of pyrrolidone, and accelerating the industrialization process of producing pyrrolidone by enzymatic conversion method.

### BRIEF DESCRIPTION OF THE DRAWINGS

FIG. 1 is an SDS-PAGE picture of the inducible expression of carnitine-CoA ligase CaiC in the present invention, in which Lane M is a low-molecular-weight protein Marker; and Lanes 1-3 are respectively band sizes of the target protein in the supernatant, the pellet and the whole cells after inducible expression with 0.2 mM IPTG at 25° C.

FIG. 2 shows verification of the enzyme activity, comparing the production of pyrrolidone with the control without various components in the transformation system.

FIG. 3 shows the relationship between pH of the transformation buffer and pyrrolidone production.

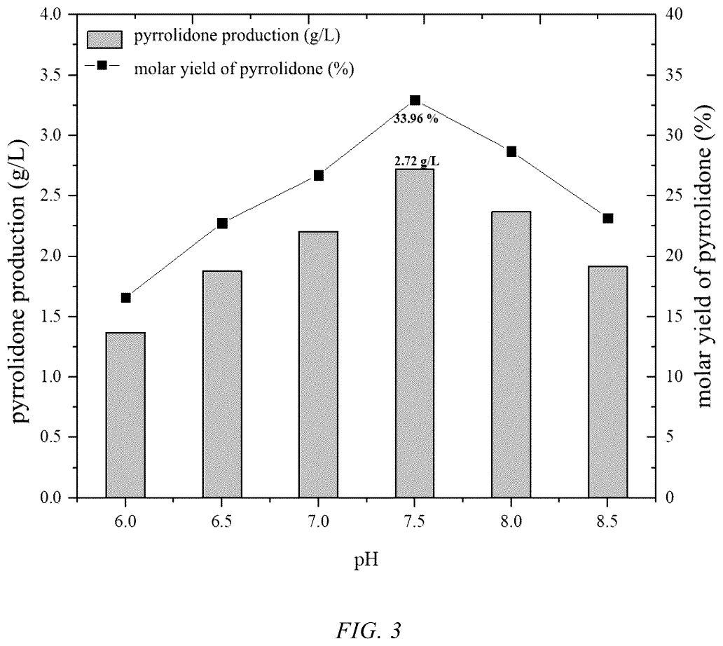

FIG. 4 shows the relationship between the Mg²⁺ concentration and pyrrolidone production.

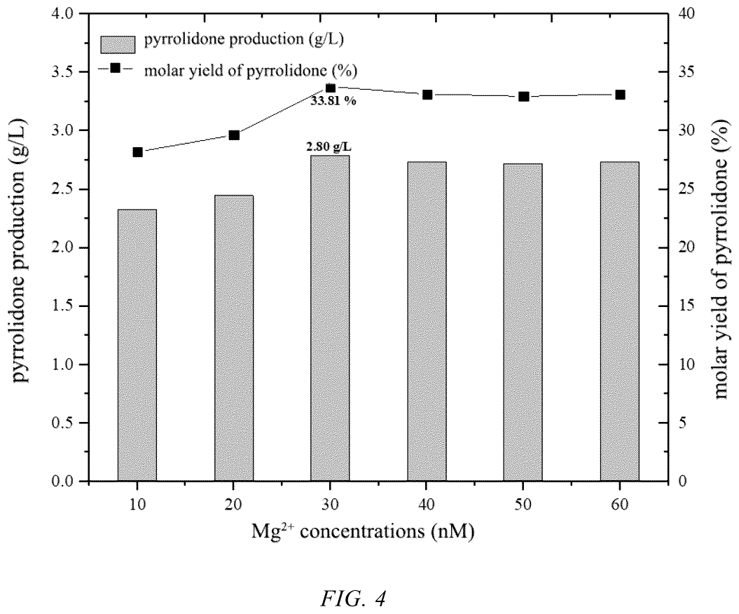

FIG. 5 shows the relationship between the transformation temperature and pyrrolidone production.

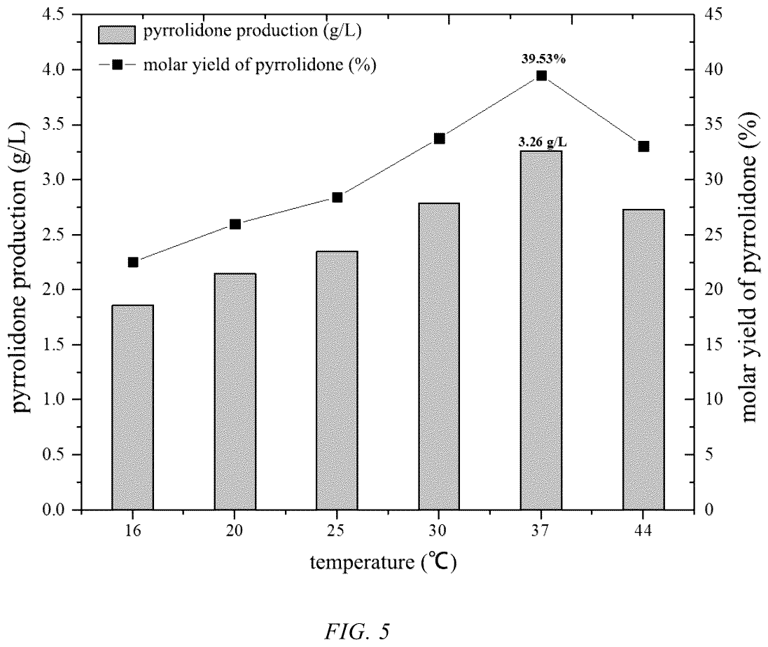

FIG. 6 shows the relationship between the substrate concentration and pyrrolidone production.

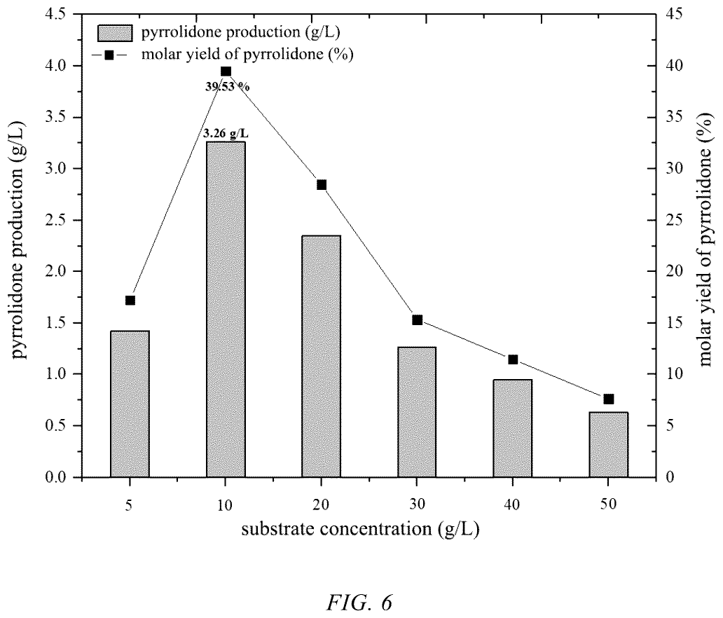

### DETAILED DESCRIPTION OF THE PREFERRED EMBODIMENTS

The present invention will be further described below in connection with specific examples, so that those skilled in the art can better understand and implement the present invention; however, the present invention is not limited thereto.

The pET-28a(+) plasmid involved in the following examples is purchased from Novagen (Madison, WI, U.S.A.), The restriction endonuclease, primeSTAR, and homologous recombinase are purchased from TaKaRa (Dalian, China). The standard γ-aminobutyric acid and pyrrolidone are both purchased from Sigma-Aldrich, and other reagents are all commercially available.

The culture media involved in the following examples include:
LB liquid medium: tryptone 10 g/L, yeast powder 5 g/L, and sodium chloride 10 g/L, sterilized at 121° C. for 20 min; and
LB solid medium: 2% agar added on the basis of LB liquid medium.

TB liquid medium: KH 2 PO₄ 2.31 g/L, K₂HPO₄·3H₂O 16.42 g/L, yeast powder 24 g/L, tryptone 12 g/L, and glycerol 4 g/L.

### Example 1: Expression and Purification of Carnitine-CoA Ligase CaiC

Construction of genetically engineered strain and expression of protein: Taking the nucleotide sequence (as shown in SEQ ID NO:2) of a target protein coding gene in Escherichia coli (strain K12) as a template and using F1 and R1 as primers (underlined are EcoR I and Hind III restriction endonuclease cleavage sites, respectively), PCR amplification was carried out. Amplification procedure:

5 min at 95° C., 29 cycles (10 s at 98° C., 15 s at 55° C., and 1.5 min at 72° C.), and 5 min at 72° C.

(SEQ ID NO: 3)
F1: agcaaatgggtcgcggatccgaattcATGGATATCATTGGCGGACA
ACATCTAC;
(SEQ ID NO: 4)
R1: tggtgctcgagtgcggccgcaagcttTTTCAGATTCTTTCTAATTA
TTTTCCCCGAGCAAT

A cDNA sequence of the carnitine-CoA ligase CaiC gene coding region was obtained. After the PCR product was collected, it was enzymatically cleaved and ligated to the pET-28a(+) plasmid vector that had been enzymatically cleaved with the same two restriction enzymes, to obtain a recombinant expression plasmid pET-28a(+)-CaiC. The recombinant plasmid pET-28a(+)-CaiC was transformed into E. coli BL21(DE3). The obtained positive engineered strain was identified by PCR, and designated as E. coli BL21/pET-28a(+)-CaiC.

The engineered strain E. coli BL21/pET-28a(+)-CaiC was inoculated into LB liquid medium, and incubated for 12 h to obtain a seed culture. The seed culture was inoculated into fresh TB liquid medium in an amount of 5% (v/v), and incubated for 2 h. IPTG at a final concentration of 0.2 mM was added, and the cells were cultured at 25° C. for 14 h, to induce the expression of the recombinant target protein. The cells were collected by centrifuging 150 mL of induced fermentation broth at 6000 r/min.

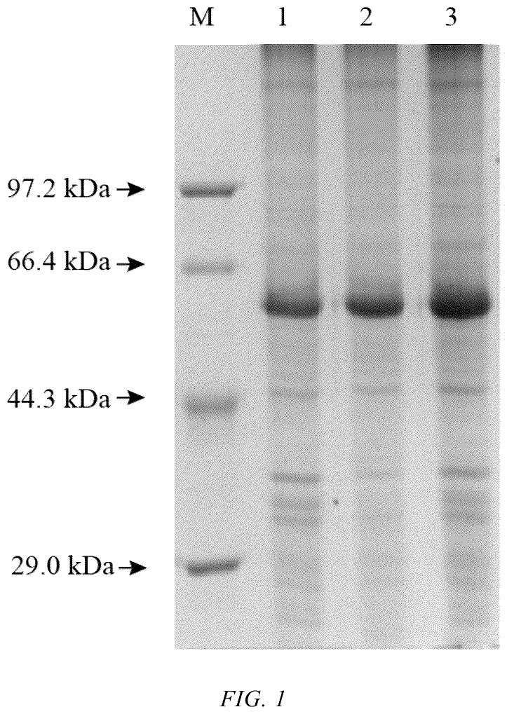

The results are shown in FIG. 1, in which Lanes 1-3 are band sizes of proteins contained in the supernatant, the pellet and the whole cells. It can be seen that the target protein is expressed in the whole cells, the supernatant and the pellet, and the band sizes are the same.

### Example 2: Activity Verification of Carnitine-CoA Ligase CaiC

Specifically, the strain E. coli BL21/pET-28a(+)-CaiC stored in a glycerin tube was spread on LB solid medium, and incubated at a constant temperature of 37° C. until single clones were grown. A single clone was picked into fresh LB liquid medium, and incubated at 200 rpm and a constant temperature of 37° C. to obtain a seed culture. The seed culture was inoculated into fresh TB liquid medium in an amount of 5% (v/v), and incubated for 2 h. IPTG at a final concentration of 0.2 mM was added, for induction culture at 25° C. for 14 h. After that, the cells were collected.

0.2 g of whole cells expressing carnitine-CoA ligase CaiC protein after induction culture, 0.1 g γ-aminobutyric acid (C4H9NO2, GABA), 500 μL of 1M ATP, 500 μL of 1M MgSO₄ and 9 mL of PBS buffer (pH7.4) were added to a 100 mL conical flask, reacted at 30° C. for 24 h, and centrifuged at 12000 r/min for 10 min. The supernatant was collected, filtered through a 0.22 μm aqueous-system filter membrane, and analyzed by HPLC.

The HPLC analysis were specifically as follows.

Agilent ZORBAX SB-C18 (5 μm, 250×4.6 mm) was used as a chromatographic column, the suction-filtered and ultrasonically degassed methanol/acetonitrile/water (5/5/90, v/v/v) was used as the mobile phase, the volume of injection was 10 μL the column temperature was 30° C., the wavelength of the UV detector was 205 nm, the flow rate was 0.5 mL/min, and the sample treatment time was 10 min. Under this detection condition, the retention time of pyrrolidone was 8.078 min.

Molar yield of pyrrolidone=(P/S₀)×100%,

where P represents the final molar concentration of pyrrolidone, and S₀ represents the initial molar concentration of γ-aminobutyric acid.

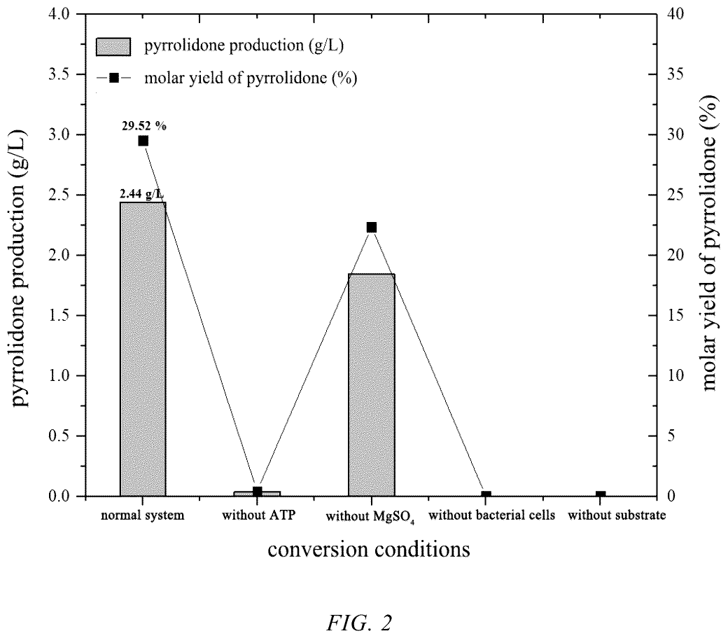

The results are specifically shown in FIG. 2. It can be seen from FIG. 2 that the catalytic effect of the whole cell is a molar yield of 29.52%. The results show that carnitine-CoA ligase CaiC has obvious cyclization activity. On the contrary, the control group without the bacterial cells or the substrate has no corresponding catalytic effect, and in the reaction system without ATP or MgSO₄, the molar yields were significantly reduced.

### Example 3: Optimum Reaction pH for Whole Cells

20 g/L whole cells, 10 g/L γ-aminobutyric acid, 50 mM ATP and 50 mM MgSO₄ were added to a 100 mL conical flask, and a PBS buffer at pH 6.0, pH 6.5, pH 7.0, pH 7.5, pH 8.0 and pH 8.5 are respectively added to form a 10 mL reaction system. The reaction was carried in a constant-temperature shaker at 30° C. and 200 rpm for 24 h. The yield of pyrrolidone was determined according to the above-mentioned detection method, and the molar yield was calculated. The result shows that the cyclization activity of carnitine-CoA ligase CaiC increases with increasing pH in the range of pH 6.0-pH 7.5, and reaches a peak at about pH 7.5, at which the yield of pyrrolidone is 2.72 g/L and the molar yield is 32.96%. The cyclization activity subsequently decreases with the further increase in pH. This indicates that the neutral environment is more favorable for the cyclization reaction catalyzed by the carnitine-CoA ligase CaiC, and whole cells have better cyclization activity at pH 7.5.

### Example 4: Optimum Mg²⁺ Concentration for Whole Cells

The specific process was shown in Example 3, except that the yield of pyrrolidone after 24 h of conversion with the carnitine-CoA ligase CaiC in the buffer pH 7.5 at various Mg²⁺ concentrations (10, 20. 30, 40, 50, 60 mM) was determined, and the molar yield was calculated. The result shows that the cyclization activity of carnitine-CoA ligase CaiC increases with increasing Mg²⁺ concentration in the range of 10-30 mM, and reaches a peak at 30 mM, at which the yield of pyrrolidone is 2.80 g/L and the molar yield is 33.81%. The cyclization activity is kept almost unchanged in the range of 30-60 mM. Therefore, a Mg²⁺ concentration of 30 mM is more favorable to the cyclization reaction catalyzed by the carnitine-CoA ligase CaiC, at which the carnitine-CoA ligase CaiC has better cyclization activity.

### Example 5: Optimum Reaction Temperature for Whole Cells

The specific process was shown in Example 3, except that the yield of pyrrolidone after 24 h of conversion with the carnitine-CoA ligase CaiC in the buffer pH 7.5 with 30 mM MgSO₄ at various temperatures (16, 20, 25, 30, 37, and 44° C.) was determined, and the molar yield was calculated. The result shows that the cyclization activity of carnitine-CoA ligase CaiC increases with increasing temperature in the range of 16-37° C., decreases with increasing temperature in the range of 37-44° C., and reaches a peak at 37° C., at which the yield of pyrrolidone is 3.26 g/L and the molar yield is 39.53%. Therefore, a conversion temperature of 37° C. is more favorable to the cyclization reaction catalyzed by the carnitine-CoA ligase CaiC, at which the carnitine-CoA ligase CaiC has better cyclization activity.

### Example 6: Optimum Substrate Concentration for Whole Cells

The specific process was shown in Example 3, except that the yield of pyrrolidone after 24 h of conversion with the carnitine-CoA ligase CaiC in the buffer pH 7.5 with 30 mM MgSO₄ at 37° C. at various concentrations (5, 10, 20, 30, 40, and 50 g/L) of substrate was determined, and the molar yield was calculated. The result shows that the cyclization activity of CaiC enzyme increases with increasing substrate concentration in the range of 5-10 g/L, decreases with increasing substrate concentration in the range of 10-50 g/L, and reaches a peak at 10 g/L, at which the yield of pyrrolidone is 3.26 g/L and the molar yield is 39.53%. Therefore, a substrate concentration of 10 g/L is more favorable to the cyclization reaction catalyzed by the carnitine-CoA ligase CaiC, and an elevated substrate concentration will produce an inhibitory effect.

The above-described embodiments are merely preferred embodiments for the purpose of fully illustrating the present invention, and the scope of the present invention is not limited thereto. Equivalent substitutions or modifications can be made by those skilled in the art based on the present invention, which are within the scope of the present invention as defined by the claims. The scope of the present invention is defined by the appended claims.

1. A method for preparing pyrrolidone, comprising:
catalytically preparing pyrrolidone from γ-aminobutyric acid in the presence of carnitine-CoA ligase CaiC or a whole cell expressing carnitine-CoA ligase CaiC.
wherein the carnitine-CoA ligase CaiC has the amino acid sequence as shown in SEQ ID NO:1,
wherein the nucleotide sequence encoding the carnitine-CoA ligase CaiC is as shown in SEQ ID NO:2, and
wherein the carnitine-CoA ligase CaiC affords a yield of pyrrolidone of 3.26 g/L and a molar yield of 39.53% in 24 hours.

2. (canceled)

3. (canceled)

4. The preparation method according to claim 1, wherein the whole cell is obtained by introducing expression of carnitine-CoA ligase CaiC in the whole cell with IPTG for 12-16 h.

5. The method according to claim 4, wherein the recombinant strain is produced with Escherichia coli as a host, and the carnitine-CoA ligase CaiC is expressed using PET-28a as an expression vector.

6. The preparation method according to claim 5, wherein the E. coli is Escherichia coli BL21 (DE3).

7. The method according to claim 1, wherein a catalytic reaction system of catalytically preparing pyrrolidone comprises γ-aminobutyric acid, ATP and Mg²⁺.

8. The method according to claim 7, wherein in the catalytic reaction system, the whole cell has a final concentration of 15-25 g/L, and γ-aminobutyric acid has a final concentration of 5-15 g/L.

9. The method according to claim 7, wherein the catalytic reaction system comprises 40-60 mM ATP and 20-40 mM Mg²⁺.

10. The method according to claim 7, wherein the catalytic reaction system has a pH of 7.4-7.6, and the reaction temperature is 35-38° C.
